(19) United States
(12) Patent Application Publication
(10) Pub. No.: US 2016/0129106 A1
(43) Pub. Date: May 12, 2016

(54) Method for preparing virosomes

(71) Applicant: Crucell Holland B.V., Leiden (NL)

(72) Inventors: Wenyu Dong, Leiden (NL); Pieter Rijken, Leiden (NL); Mike Ugwoke, Leiden (NL)

(21) Appl. No.: 14/998,169

(22) Filed: Dec. 23, 2015

Related U.S. Application Data
(63) Continuation of application No. PCT/EP2014/063814, filed on Jun. 30, 2014.

(30) Foreign Application Priority Data
Jul. 2, 2013 (EP) 13174696.8

(51) Int. Cl. A61K 39/145; C12N 7/00

(52) U.S. Cl. CPC A61K 39/145; C12N 7/00; C12N 2760/16134; C12N 2760/16151; C12N 2760/16234; C12N 2760/16251; C12N 2760/16161; C12N 2760/16261; A61K 2039/5252

(57) ABSTRACT

This disclosure relates to methods for preparing a virosome preparation comprising viral envelope protein of at least two, preferably at least three, more preferably at least four, different enveloped viruses, as well as to virosomal preparations obtained thereby. The disclosure also relates to the use of the virosomal preparations, e.g., as a vaccine.

### CROSS-REFERENCE TO RELATED APPLICATIONS

This application is a continuation of International Patent Application PCT/EP2014/063814, filed Jun. 30, 2014, designating the United States of America and published in English as International Patent Publication WO 2015/000832 Al on Jan. 8, 2015, which claims the benefit under Article 8 of the Patent Cooperation Treaty and under 35 U.S.C. §119(e) to European Patent Application Serial No. 13174696.8, filed Jul. 2, 2013, the disclosure of each of which is hereby incorporated herein in its entirety by this reference.

### TECHNICAL FIELD

The disclosure relates to the field of medicine and, in particular, to the field of infectious diseases. More in particular, the disclosure relates to methods for preparing virosome preparations and to the virosome preparations obtainable thereby. The disclosure furthermore relates to novel virosomes, to immunogenic compositions that include the virosomes, and to uses thereof, e.g., in vaccines.

### BACKGROUND

Virosomes are reconstituted phospholipid (PL) membranes containing proteins from a virus, like hemagglutinin (HA) and neuraminidase (NA) from an influenza virus. These virosomes are derived from influenza viruses, or from other enveloped viruses, such as, but not limited to, the following families: flaviviridae (e.g., Dengue virus, Hepatitis C virus HEV, Japanese encephalitis virus, Yellow fever virus, West Nile virus), Poxviridae (e.g., Cowpox virus, Monkeypox virus, vaccinia virus, Variola virus), Retroviridae (e.g., immunodeficiency viruses HIV/SIV), paramyxoviridae (e.g., Measles virus, Mumps virus, Parainfluenza viruses, metapneumovirus, Respiratory Syncytial virus RSV), and Orthomyxoviridae (e.g., influenza viruses). Virosomes generally are prepared from such enveloped viruses by solubilization of the viral envelope and removal of the nucleocapsid containing the viral genome, followed by reconstitution of an “empty” viral envelope comprising a lipid membrane containing viral glycoproteins derived from the enveloped virus, such as hemagglutinin (HA) in the case of influenza virosomes. Virosomes can be produced by either the addition of exogenous lipids (e.g., as described in US 2009/0263470 and US 2009/0087453) or without the addition of exogenous lipids (as described in U.S. Pat. No. 7,901,920). Additional antigens may be adhered to the virosomes or cross-linked to lipid molecules in order to anchor them in the virosomal membrane.

Since virosomes do not contain the viral genomic material (e.g., viral genomic RNA or DNA), they are nonreplicative in nature, which make them safe for administration to animals and humans in the form of an immunogenic composition (e.g., as a vaccine), or as an adjuvant. Virosomes thus have been especially useful in the field of vaccination, where it is desired to stimulate an immune response to an antigen associated with a particular disease or disorder. In such cases, an antigen is typically encapsulated in or bound to the virosome, which then delivers this antigen to the host immune system to be vaccinated. By virtue of the particular antigen delivered, the resulting prophylactic and/or therapeutic effect is necessarily specific for the disease or disorder with which the antigen is associated. Thus, influenza virosomes that essentially are reconstituted virus envelopes containing the hemagglutinin antigen incorporated in a phospholipid membrane envelope, but without having the genetic background genome of the influenza virus itself, have been approved for seasonal influenza vaccination (marketed under the trademark INFLEXAL® V).

Infections with influenza viruses are associated with a broad spectrum of illnesses and complications that result in substantial worldwide morbidity and mortality, especially in the elderly and patients with chronic illnesses. Vaccination against influenza is most effective in preventing the possible fatal complications associated with this infection, and much effort has been put in the development of influenza vaccines, including those based on virosome technology. The yearly trivalent influenza vaccine typically contains antigens from two influenza A virus strains and one influenza B virus strain. Based on the epidemiological surveys by over one hundred National Influenza Centers worldwide, the World Health Organization (WHO) yearly recommends the composition of the influenza vaccine, usually in February for the Northern hemisphere and in September for the Southern hemisphere. In this context, this practice limits the time window for production and standardization of the influenza vaccine.

The current process for the manufacture of the trivalent virosomal influenza vaccine involves multiple processes, which make the process less efficient and increase the cost of goods. In addition, due to the fact that multiple process steps are involved, there is a relatively high risk of contamination occurring during the manufacturing process. Furthermore, due to the increasing world population, growing and emerging economies, intensified international traveling, the growing spread of yearly influenza epidemics, and the threat of worldwide influenza pandemics, the demand for influenza vaccines is still growing. There is, thus, an ongoing need for simplified and/or improved methods for preparing virosome preparations.

### BRIEF SUMMARY

This disclosure provides a novel method for preparing a virosome preparation comprising a viral envelope protein of at least two, preferably at least three, more preferably at least four, different enveloped viruses. The method of the disclosure comprises the steps of:
(a) providing at least two, preferably at least three, more preferably at least four, enveloped viruses and optionally inactivating the at least two, preferably at least three, more preferably at least four, enveloped viruses;
(b) solubilizing the at least two, preferably at least three, more preferably at least four, viral envelopes by adding a solubilizing agent; and
(c) reconstituting virosomal membranes comprising the viral envelope protein of the at least two, preferably at least three, more preferably at least four, different viruses by removing the solubilizing agent,
wherein the viral envelopes of the at least two, preferably at least three, more preferably at least four, different viruses are blended prior to reconstitution of the virosomal membranes.

The disclosure further provides virosome preparations obtainable by this method.

This disclosure further provides virosomes having a virosomal membrane comprising viral envelope protein of at least two, preferably at least three, more preferably at least four, different viruses.

The enveloped virus preferably is an influenza virus, and the at least two, three, or four different viruses preferably are two, three, four or more different influenza A and/or B virus strains. However, the method of the disclosure also applies to all other enveloped viruses that can be used to prepare virosomes.

The disclosure also provides compositions, preferably immunogenic compositions, comprising the virosome preparations or virosomes.

The disclosure also relates to (immunogenic) compositions comprising the virosomes or virosomal preparations, and the use thereof, e.g., for eliciting and/or increasing an immune response. In a preferred embodiment, the composition is a vaccine. The compositions are preferably suitable for human administration. The compositions may be used in methods of preventing and/or treating any viral disease, in particular, but not limited to, those caused by influenza virus. The disclosure further relates to the use of the virosomes as a vaccine and/or as an adjuvant. The disclosure, in particular, relates to the use of the virosomes as a vaccine for the prevention and/or treatment of influenza.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a process flow chart showing the procedure of preparing monovalent virosome particles of the prior art.

FIG. 2 shows blending of three monovalent virosomal preparations into a trivalent virosomal preparation according to the prior art.

FIG. 3 is a process flow chart for the preparation of a virosome preparation according to the disclosure.

FIGS. 4A and 4B show the virosome particles prepared using the method of the prior art (FIG. 4A) and using the method of this disclosure (FIG. 4B).

FIG. 5 shows a CryoTEM micrograph of cocktail virosomes made according to the current disclosure, showing optically dense regions in the virosome membrane indicating regions of protein (HA and NA) insertion in the membranes.

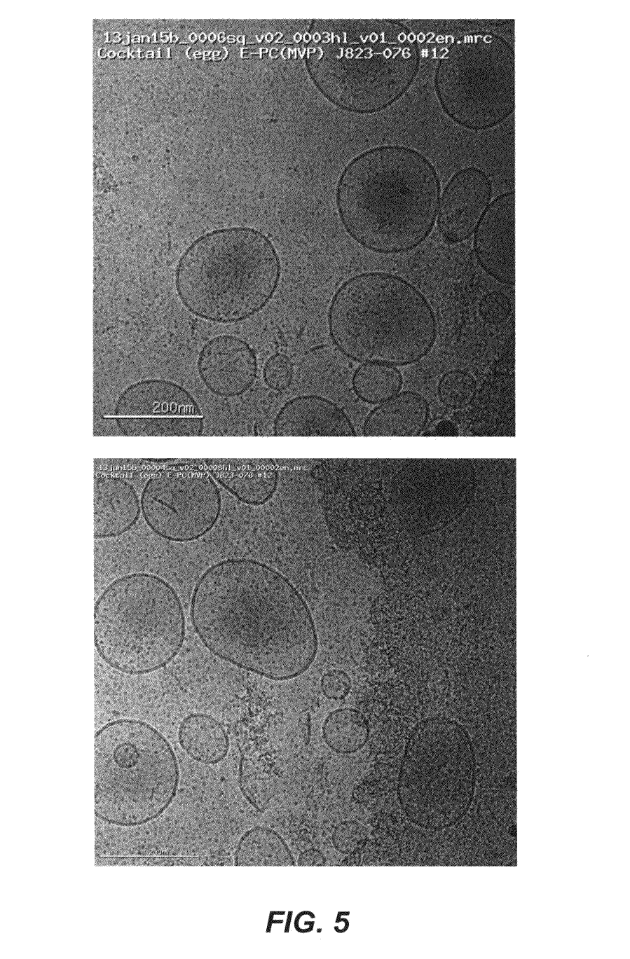

### DETAILED DESCRIPTION

This disclosure provides a novel and improved method for preparing virosome preparations comprising viral envelope protein of at least two, preferably at least three, more preferably at least four, different enveloped viruses, comprising the steps of:
(a) providing at least two, preferably at least three, more preferably at least four, enveloped viruses and optionally inactivating the at least two, preferably at least three, more preferably at least four, enveloped viruses;
(b) solubilizing the at least two, preferably at least three, more preferably at least four, viral envelopes by adding a solubilizing agent; and
(c) reconstituting virosomal membranes comprising viral envelope protein of the at least two, preferably at least three, more preferably at least four, different viruses by removing the solubilizing agent,
wherein the viral envelopes of the at least two, preferably at least three, more preferably at least four, different viruses are blended prior to reconstitution of the virosomal membranes.

According to the disclosure, a simplified method for preparing multivalent virosomal preparations, in particular, virosome preparations comprising virosomes having a virosomal membrane comprising viral envelope protein of two or more different enveloped viruses, in particular, viral envelope protein of at least three, preferably at least four, different enveloped viruses is provided. The term “virosomal membrane” as used herein refers to a membrane structure that is reconstituted in vitro and that is composed of a lipid bilayer with integrated viral envelope proteins. The term “envelope protein” refers to protein that is encoded by an enveloped virus and that is normally associated with or embedded in the viral envelope.

In certain embodiments, the method further comprises a step of adding exogenous lipids. By “exogenous lipids” are meant lipids that are not endogenous to enveloped virus but that are added to the virus components during preparation of the virosomes. According to the disclosure, a broad range of lipids can be incorporated in the virosomal membrane. The preferred virosomal preparations are based on phospholipids, such as, but not limited to, egg-derived phospholipids in order to minimize the complexity of the formulation.

In certain embodiments, the method further comprises the step of adding further exogenous components. The term “exogenous component” according to the disclosure means any additional component that is not endogenous to the virus but is added to the virus components during preparation of the virosomes. Exogenous components that may be added according to the disclosure include lipoproteins, lipopolysaccharides, adjuvants and/or targeting ligands.

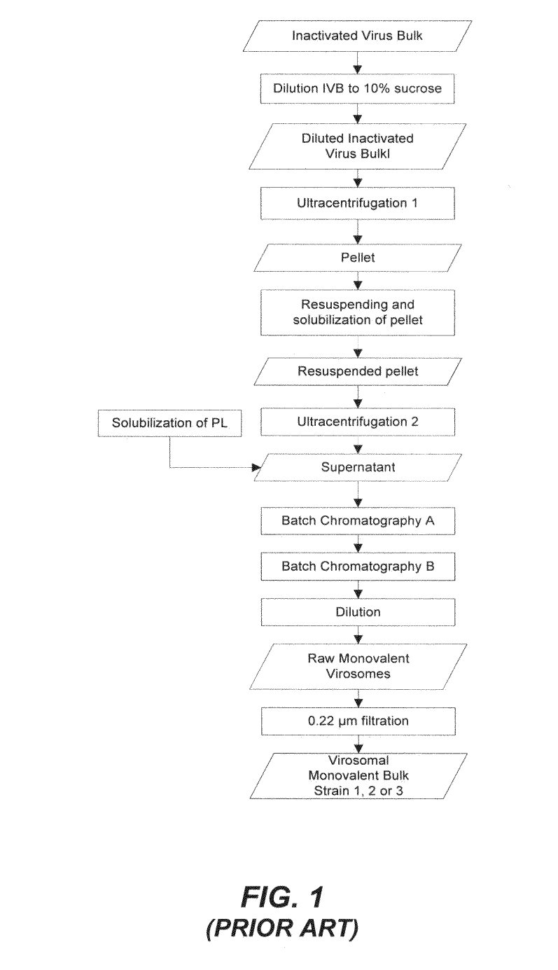

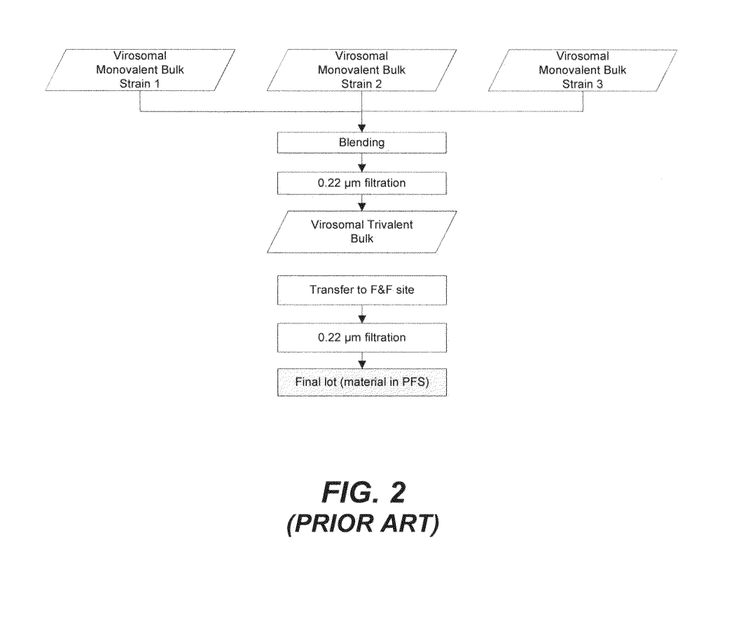

Multivalent virosomal compositions are known, i.e., vaccine compositions containing different virosomes, each with the viral envelope proteins of a single virus strain, such as the virosomal influenza vaccine INFLEXAL V®, which typically contains hemagglutinin (HA) from two influenza A virus strains, generally an influenza A H1N1 and an H3N2 strain and an influenza B strain. Such multivalent virosomal compositions typically are produced by preparing monovalent virosome preparations from the different influenza virus strains and subsequently blending the monovalent virosome preparations into a multivalent virosome preparation. FIG. 1 schematically shows the current process steps for producing an influenza virosomal vaccine. In short, the virosomal vaccine is produced by first making individual virosome monovalent bulk (VMVB) from each virus strain by multiple steps. Subsequently, the VMVBs from different virus strains are blended into trivalent virosome preparations (three strains), or optionally quadrivalent preparations (four strains), prior to filling in final containers as the final drug product (FIG. 2).

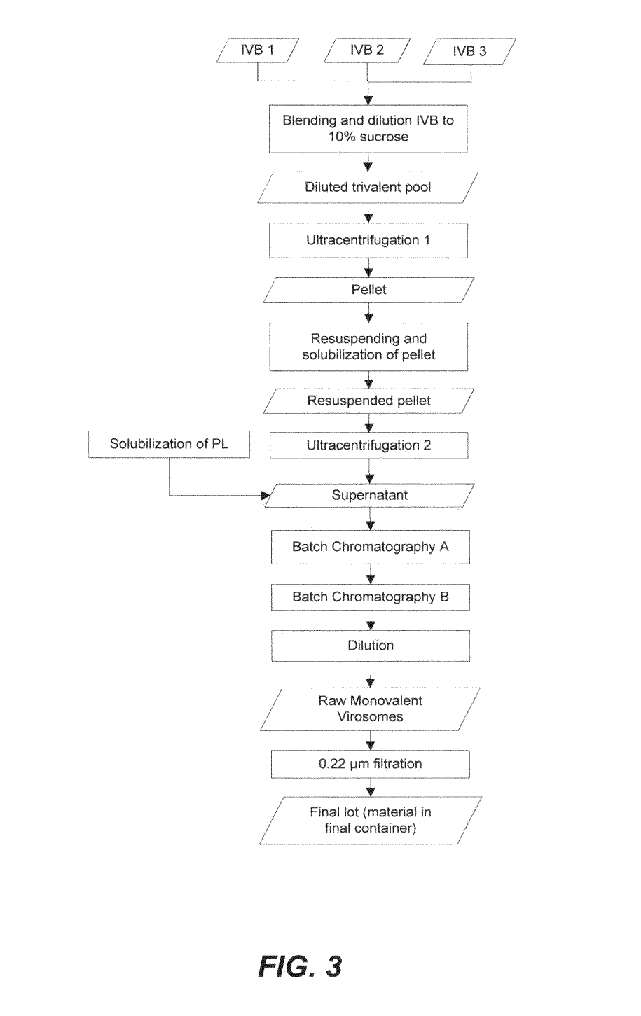

According to the disclosure, it has surprisingly been shown that this method could be simplified substantially by combining several process flows into one. Thus, as shown in FIG. 3, whole viruses, such as influenza viruses (either activated or inactivated), or the supernatants after solubilization of the viral membranes with solubilizing solutions, of two, three, four, or more different viruses or virus strains are blended. Subsequently, the blended virus bulk or the supernatant can be processed in one single flow to achieve the final multivalent virosomal preparation, such as, e.g., a trivalent or quadrivalent virosome product. Prior to the disclosure, it was generally thought by people skilled in the art that the method as described herein could lead to lop-sided incorporation of HA into the bilayer. In addition, the different stability of different influenza strains could lead to differences in stability during processing and, thus, to product of less quality. Surprisingly, however, it was demonstrated that a random and equitable distribution of HA from different virus strains per virosome particle was obtained, and no negative effect on product quality was observed.

The influenza viruses may be inactivated. According to the disclosure, inactivation of the viruses may be accomplished using methods well known in the art, e.g., by use of formaldehyde or beta-propiolactone (BPL).

Solubilization of the viral envelopes is achieved by adding a solubilizing agent. Suitable solubilizing agents are known in the art. Preferably, the solubilizing agent is a non-ionic detergent. Examples for non-ionic detergents according to the disclosure comprise detergents, e.g., such as octaethylene glycol mono(N-dodecyl)ether (OEG), TRITON® X-100, TRITON® X-114, NP 40 and, Polysorbate 20 or 80. The detergents may be preferably used in a concentration range of 10 mM to 1000 mM. In certain embodiments, the non-ionic detergent is octaethylene glycol monododecyl ether (OEG).

After solubilization of the viral envelopes and, optionally, addition of exogenous lipids, and/or further exogenous components such as, but not limited to, adjuvants and targeting ligands, the virosomal membranes are reconstituted by removing the solubilizing agent. Removing the solubilizing agent according to the disclosure may be accomplished by processes such as dialysis, diafiltration, or chromatography. The latter, chromatography, which is based on eliminating detergent by adsorbance to a matrix (e.g., beads, resin), is preferred.

In certain embodiments, the viral envelopes of the at least two, preferably at least three, more preferably at least four, enveloped viruses are blended prior to inactivating the at least two, three, or four enveloped viruses. In such embodiments, the different whole viruses are thus blended and subjected to a simultaneous virus inactivation step.

In a further embodiment, the viral envelopes of the at least two, preferably at least three, more preferably at least four, enveloped viruses are blended after inactivation of the viruses and prior to solubilizing the viral envelopes. The individual viruses are thus inactivated individually prior to blending them.

In yet another embodiment, the viral envelopes of the at least two, preferably at least three, more preferably at least four, enveloped viruses are blended after (separately) solubilizing the viral envelopes. Thus, preparations comprising solubilized viral envelopes of the at least two, preferably at least three, more preferably at least four, different viruses are blended prior to reconstitution of the viral envelopes.

Viruses, such as, e.g., influenza viruses, generally contain several virus strains that usually result from mutations and that may show different pathogenic profiles. These different strains can be combined in order to provide multivalent, e.g., trivalent, vaccines. In certain embodiments, the at least two, preferably at least three, more preferably at least four, different enveloped viruses thus comprise at least two, preferably at least three, more preferably at least four, different virus strains of a specific type or species of an enveloped virus. In a particular embodiment, the enveloped virus is an influenza virus and the envelope protein is hemagglutinin (HA) and/or neuraminidase (NA). The at least two, three, or four virus strains thus may comprise several influenza A virus strains, such as, e.g., H1N1 and/or H3N2 influenza virus strains, and/or several influenza B virus strains. Other enveloped viruses may, however, also be used according to this disclosure.

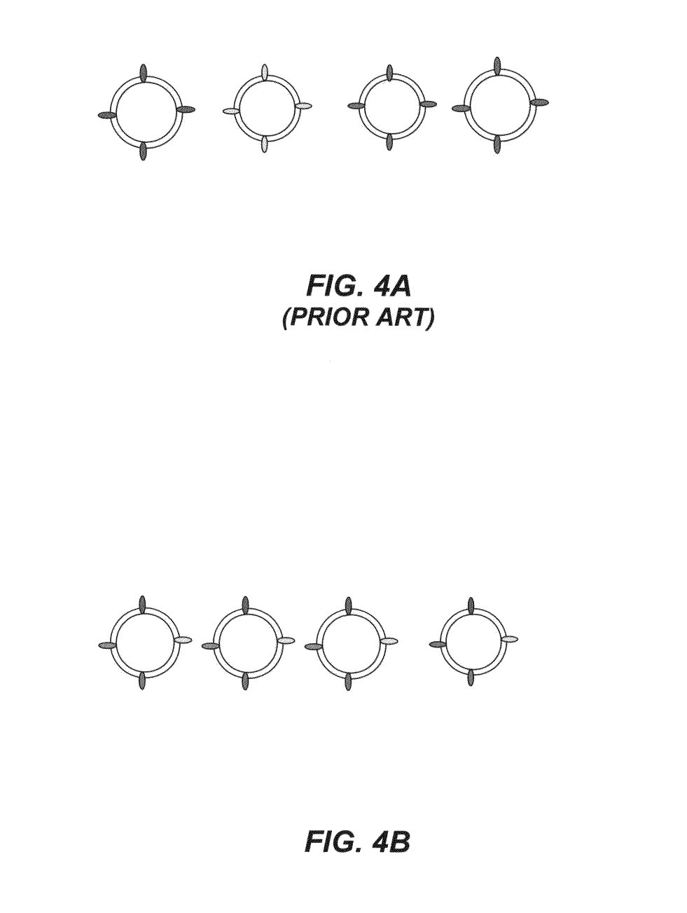

According to this disclosure, it has been shown that the structure of the virosome particles obtained by the method described herein is different from the virosome particles obtained using methods of the prior art. Thus, envelope protein (e.g., HA) from different viruses or virus strains is spiked in the same virosome particle (FIG. 4B), yielding a homogenous distribution of envelope protein (e.g., HA) from different viruses or virus strains among all virosome particles. This is contrary to the current situation involving blending of, e.g., three or four different virosome preparations, wherein the final virosomal preparation is a heterogeneous mixture of three or four monovalent virosome types (FIG. 4A). Since in vitro stability of the final multivalent virosome preparations generally is virus strain-dependent, this needs to be improved for each virus strain in order to have a similar stability profile. With the method according to the disclosure, a stable virosome preparation is obtained. Virosome preparations obtainable by the method of this disclosure thus also form part of this disclosure.

In certain embodiments, the method may further comprise a step of adding one or more additional antigens. The antigen(s) may be incorporated into the virosome (e.g., contained in its lumen), absorbed to/bound to the surface of the virosome, integrated into the lipid membrane of the virosome, and the like.

In certain embodiments, the method further comprises the step of purifying the reconstituted virosomes. Purification of the virosomes may be accomplished using standard techniques known in the art, such as, but not limited to, batch chromatography with, e.g., BIOBEADS®, affinity chromatography, gel filtration, dialysis, cross-flow filtration, etc.

In certain embodiments, the method further comprises the step of size reduction of the reconstituted virosomes. Size reduction may be accomplished by techniques, such as, but not limited to, (high pressure) homogenization, and extrusion.

The disclosure further relates to virosome particles having a virosomal membrane comprising viral envelope protein of at least two, preferably at least three, more preferably at least four, different enveloped viruses. In a preferred embodiment, the virosomes comprise envelope protein of at least two, preferably at least three, more preferably at least four, different virus strains of an enveloped virus. In a particular embodiment, the enveloped virus is an influenza virus and the viral envelope protein is HA and/or NA.

In certain embodiments, the virosomes comprise envelope protein of at least three different virus strains, in particular, at least three different influenza virus strains, such as, e.g., two influenza A virus strains, such as, e.g., H1N1 and/or H3N2 influenza virus strains, and one influenza B virus strain. In another embodiment, the virosomes comprise envelope protein of at least four different virus strains, in particular, at least four different influenza virus strains, such as, e.g., two influenza A virus strains, such as, e.g., H1N1 and/or H3N2 influenza virus strains, and two influenza B virus strains.

In further embodiments, the virosomes further comprise one or more antigens. Thus, the virosomes may comprise one or more additional antigens incorporated in the virosomal membrane and/or linked thereto. The antigen may be incorporated into the virosome (e.g., contained in its lumen), absorbed to/bound to the surface of the virosome, integrated into the lipid membrane of the virosome, and the like. A virosome loaded with antigen may be used as an antigen delivery vehicle.

The disclosure furthermore relates to compositions comprising the above-described virosome preparations or virosomes. The disclosure further provides immunogenic compositions comprising a therapeutically effective amount of the virosome preparations or virosomes. The (immunogenic) compositions may further comprise a pharmaceutically acceptable excipient. By “pharmaceutically acceptable excipient” is meant any inert substance that is combined with an active molecule for preparing an agreeable or convenient dosage form. The “pharmaceutically acceptable excipient” preferably is an excipient that is non-toxic to recipients at the used dosages and concentrations, and is compatible with other ingredients of the formulation. Pharmaceutically acceptable excipients are widely applied and known in the art (see Remington's Pharmaceutical Sciences, 18th edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]). The exact formulation should suit the mode of administration.

In certain embodiments, the immunogenic compositions may comprise, or are administered in combination with, an adjuvant. The adjuvant may be administered before, concomitantly with, or after administration of the composition. Examples of suitable adjuvants include saponin formulations, such as, but not limited to, QS21, immunostimulating complexes (ISCOMs) (see, e.g., U.S. Pat. No. 5,057,540; WO 90/03184, WO 96/11711, WO 2004/004762, WO 2005/002620); bacterial or microbial derivatives, examples of which are monophosphoryl lipid A (MPL), 3-O-deacylated MPL (3 dMPL), CpG-motif containing oligonucleotides, ADP-ribosylating bacterial toxins or mutants thereof, such as E. coli heat labile enterotoxin LT, cholera toxin CT, pertussis toxin PT, or tetanus toxoid TT, Matrix M and cationic lipids. However, since virosomes have inherent adjuvanting activity, in certain embodiments, the immunogenic compositions of this disclosure do not comprise additional adjuvants.

The disclosure also relates to methods for inducing and/or increasing an immune response in a subject, the method comprising administering to a subject the above-described virosomal preparations or immunogenic compositions. According to the disclosure, the induced immune response may be directed against the viral envelope protein and/or to the additional (viral) antigen(s). A subject according to the disclosure preferably is a mammal that is capable of being infected with an infectious disease-causing virus, in particular, an influenza virus. Preferably, the subject is a human subject.

In certain embodiments, the disclosure provides methods for inducing an immune response to an influenza virus envelope protein, such as HA and/or NA. In some embodiments, the immune response induced is effective to prevent and/or treat an influenza virus infection.

The term “therapeutically effective amount” as described herein refers to an amount of virosomes that is effective for preventing, ameliorating and/or treating a condition resulting from infection with a virus, in particular, but not limited to, an influenza virus. “Amelioration” as used herein may refer to the reduction of visible or perceptible disease symptoms, viremia, or any other measurable manifestation of the viral infection, in particular, the influenza infection. Prevention encompasses inhibiting or reducing the spread of virus or inhibiting or reducing the onset, development or progression of one or more of the symptoms associated with infection with the virus, in particular, influenza virus.

The disclosure also relates to the above-described compositions and/or immunogenic compositions for inducing and/or increasing an immune response in a subject, in particular, for use as a vaccine and/or as an adjuvant. The virosomal preparations, virosome particles and/or immunogenic compositions thus may be used to elicit neutralizing antibodies against the viral envelope protein, e.g., to HA and/or NA of influenza virus and/or to the additional antigen(s). The disclosure, in particular, relates to the virosomal preparations, virosomes and/or immunogenic compositions as described above for use as a vaccine in the prevention and/or treatment of a disease or condition caused by an influenza A and/or B virus.

Administration of the virosomal preparations and/or (immunogenic) compositions according to the disclosure can be performed using standard routes of administration. Non-limiting examples include parenteral administration, such as intravenous, intradermal, transdermal, intramuscular, subcutaneous, or mucosal administration, e.g., intranasal, oral, and the like. In certain embodiments, the virosomal preparations and/or immunogenic compositions are administered more than one time, i.e., in a so-called homologous prime-boost regimen. In certain embodiments where the virosomal preparations and/or immunogenic compositions are administered more than once, the administration of the second dose can be performed after a time interval of, for example, one week or more after the administration of the first dose, two weeks or more after the administration of the first dose, three weeks or more after the administration of the first dose, one month or more after the administration of the first dose, six weeks or more after the administration of the first dose, two months or more after the administration of the first dose, three months or more after the administration of the first dose, four months or more after the administration of the first dose, etc., up to several years after the administration of the first dose of the virosomal preparations and/or immunogenic compositions. It is also possible to administer the virosomal preparations and/or immunogenic compositions more than twice, e.g., three times, four times, etc., so that the first priming administration is followed by more than one boosting administration. In other embodiments, the virosomal preparations and/or immunogenic compositions according to the disclosure are administered only once. The virosomal preparations and/or immunogenic compositions may also be administered, either as prime, or as boost, in a heterologous prime-boost regimen.

In certain embodiments, prevention and/or treatment may be targeted at patient groups that are susceptible to infection with the virus, in particular, to influenza virus infection. Such patient groups include, but are not limited to, e.g., the elderly (e.g., ≧50 years old, ≧60 years old, and preferably ≧65 years old), the young (e.g., ≦5 years old, ≦1 year old), hospitalized patients and patients who have been treated with an antiviral compound but have shown an inadequate antiviral response.

In other embodiments, the virosomal preparations and/or immunogenic compositions may be administered to a subject in combination with one or more other active agents, such as existing vaccines, monoclonal antibodies and/or antiviral agents, and/or antibacterial, and/or immunomodulatory agents. In another embodiment, other active agents can be incorporated into the virosome core or bilayer.

This disclosure is further illustrated in the following Examples.

### EXAMPLE 1

### Preparation of Trivalent Influenza Virosomal Preparation According to the Disclosure

Egg-based and PER.C6® cell-based trivalent virosomal preparations were prepared according to the disclosure, after blending inactivated viruses.

### Solubilization of the Inactivated Virus Bulk and Phospholipids

The inactivated virus bulks (IVB, A. California, A. Victoria and B. Brisbane) were pooled, diluted with phosphate-buffered saline (PBS, pH 7.4), mixed with a magnetic stirrer and divided into ultracentrifugation bottles, followed by ultracentrifugation at >15000 rpm for 1 hour. In this example, the experimental starting ratio of the IVBs was 0.2:0.4:0.3 for A. California, A. Victoria and B. Brisbane, respectively. The supernatants of all the UC bottles/tubes were discarded and OEG solution (55% of required volume) serially added to resuspend the pellet in different bottles. The ultracentrifugation bottles/tubes were rinsed serially with additional OEG solution (35% of required volume), and pooled with the first suspension and the resuspended pellet stirred overnight at 4° C. The resuspended pellet was divided in the ultracentrifugation bottles/tubes and each bottle/tube was centrifuged at 28,000 rpm or higher. Gently, the supernatant was withdrawn from the bottle/tube and poured into a clean glass bottle without disturbing the pellet in the bottom of the tube. The phospholipids (PL) were weighed directly into a clean glass vial and the OEG solution (10% of required volume) added and mixed with magnetic stirrer overnight at 4° C. for solubilization. The PL suspension and supernatant containing viral envelope material were combined and mixed for 30 to 60 minutes until the mixture was free of any visible particles.

### Preparation of BIOBEADS®

The BIOBEADS® were weighed into a glass flask, methanol added and stirred for 10 minutes followed by filtration to remove the methanol. This was repeated with demineralized water. Residual moisture content of 1 gram of BIOBEADS® was determined using the Halogen Moister Analyzer 20085. Thereafter, the BIOBEADS® were subdivided into two bottles (bottle A and bottle B), water added to them and the bottles autoclaved, and subsequently stored at 2° C. to 8° C. until used.

Virosome Formation by OEG Removal with BIOBEADS®

Water was removed from batch chromatography bottle A using a syringe and needle. The supernatant with PL was transferred into bottle A, placed in a turbula shaker and shaken at 23 rpm for 1 hour at room temperature to remove the OEG.

Water was removed from batch chromatography Bottle B as well. The suspension from bottle A was transferred to bottle B and the bottle placed in the turbula shaker and shaken at 23 rpm for 1 hour at RT to complete removal of the OEG. The virosomes formed (raw trivalent virosome) were collected, the HA content determined and thereafter diluted with PBS accordingly to yield HA concentration of ˜33 μg/mL. The trivalent virosome final bulk was filtrated using 0.22 μm syringe filter and stored at 2° C. to 8° C. until used.

Cryo-TEM pictures of the obtained virosomes showed optically dense regions in the membrane indicating antigen incorporation into the virosomal membrane.

### EXAMPLE 2

### Stability Testing

The virosomal trivalent final bulk was filled (50 mL/bag) in six FLEXBOY® bags (Sartorius A G) and one glass bottle (50 mL) and stored at 2° C. to 8° C. up to three months. The samples were collected from the glass vial and six FLEXBOY® bags after three months' storage at 2° C. to 8° C. and analyzed (Table 1) where it was observed that the stability was not changed during this period.

TABLE 1
Stability study of trivalent virosome cocktail

| | | | T = 3 months |
|---|---|---|---|
| Assay | Product property | T = 0 | (2° C.-8° C.) |
| Appearance | Visual appearance | Slightly | Slightly |
| | | opalescent, | opalescent, |
| Particle Size | Particle size (nm) | 183 | 188 |
| Charge | Zeta potential (mV) | −8 | −8 |
| HPLC | HA content (μg/ml) | B. Bris. 53 | B. Bris. 59 |
| HA-SRID | HA content/in vitro | 39 (A. Cal.); | 44 (A. Cal.); |
| | potency (μg/ml) | 89 (A. Vic.); | 83 (A. Vic.); |
| | | 62 (B. Bris)* | 61 (B. Bris)* |
| | HA ratio | A. Cal., 0.2: | A. Cal., 0.2: |
| | | A. Vic., 0.5: | A. Vic., 0.4: |
| | | B. Bris, 0.3 | B. Bris, 0.3 |

*The difference in ratio was due to the difference in starting ratio of the IVBs, which was theoretical: 0.2:0.4:0.3, respectively, for A. Cal., A. Vic., and B. Bris.

1. A method for preparing a virosome preparation comprising viral envelope protein of at least two different enveloped viruses, the method comprising:
solubilizing at least two viral envelopes by adding a solubilizing agent; and
reconstituting virosomal membranes comprising viral envelope protein of said at least two different viruses by removing the solubilizing agent,
wherein the viral envelopes of said at least two different virus are blended prior to reconstitution of the virosomal membranes.

2. The method according to claim 1, wherein the viral envelopes preferably are blended prior to inactivation of the at least two viral envelopes.

3. The method according to claim 1, wherein the viral envelopes are blended prior to solubilizing the viral envelopes.

4. The method according to claim 1, wherein the viral envelopes are blended after solubilizing the viral envelopes.

5. The method according to claim 1, wherein the at least preferably two viral envelopes comprise at least two different virus strains of an enveloped virus.

6. The method according to claim 5, wherein the enveloped virus is an influenza virus and the viral envelope protein is hemagglutinin (HA) and/or neuraminidase (NA).

7. The method according to claim 1, further comprising:
one or more exogenous components to the solubilized viral envelopes.

8. A virosome preparation produced by the method of claim 1.

9. A virosome having a virosomal membrane comprising:
viral envelope of at least two different enveloped viruses.

10. The virosome according to claim 9, wherein the at least two different enveloped viruses comprise at least two different virus strains of an enveloped virus.

11. The virosome according to claim 9, wherein the enveloped virus is an influenza virus and the viral envelope protein is HA and/or neuraminidase (NA).

12. The virosome according to claim 9, further comprising:
one or more antigens incorporated and/or linked to the virosomal membrane.

13. A composition comprising:
the virosome preparation according to claim 8.

14. A composition comprising:
the virosome according to claim 9.

15. A vaccine comprising:
an amount of the composition according to claim 13 for use as a vaccine.

16. An immunogenic composition comprising:
an amount of the composition according to claim 13 for use as an adjuvant.

17. The composition according to claim 14, which is therapeutic.

18. A targeting ligand comprising:
the composition according to claim 13 for use as targeting ligand.

19. The method according to claim 1, wherein:
the virosome preparation comprises viral envelope protein of at least three different enveloped viruses; and
at least three viral envelopes are solubilized by adding a solubilizing agent.

20. The method according to claim 19, wherein:
the virosome preparation comprises viral envelope protein of at least four different enveloped viruses; and
at least four viral envelopes are solubilized by adding a solubilizing agent.

21. The method according to claim 1, further comprising:
inactivating the enveloped viruses.

22. The virosome of claim 9, having a virosomal membrane comprising:
viral envelope protein of at least three different enveloped viruses.

23. The virosome of claim 22, having a virosomal membrane comprising:
viral envelope protein of at least four different enveloped viruses.

24. The virosome of claim 22, wherein the at least three different enveloped viruses comprise at least three different virus strains of an enveloped virus.

25. The virosome of claim 23, wherein the at least four different enveloped viruses comprise at least four different virus strains of an enveloped virus.
